SEQUENCE LISTING (15 sequences)

SEQ ID NO: 1 - DNA, 23 bp, Artificial Sequence
  CG Forward Primer (forward primer for PCR of C. glutamicum)
aagctccagccacccaaaactac

SEQ ID NO: 2 - DNA, 23 bp, Artificial Sequence
  cg reverse primer (reverse primer for pcr of c. glutamicum)
ctaccaaccactaatgcgtcgtc

SEQ ID NO: 3 - DNA, 22 bp, Artificial Sequence
  CG Probe (probe for PCR of C. glutamicum)
atcgccttccagacgctcaacg

SEQ ID NO: 4 - DNA, 24 bp, Artificial Sequence
  BS Forward Primer (forward primer for PCR of B. subtilis)
acatcttaccgcaactacgaccat

SEQ ID NO: 5 - DNA, 24 bp, Artificial Sequence
  BS Reverse Primer (reverse primer for PCR of B. subtilis)
tagcatagtctttgtcccaccgta

SEQ ID NO: 6 - DNA, 23 bp, Artificial Sequence
  BS Probe (probe for PCR of B. subtilis)
ggaggcgatctatgtcttgtcca

SEQ ID NO: 7 - DNA, 23 bp, Artificial Sequence
  fr Forward Primer (forward primer for PCR of E. coli phage fr)
cttctgatccgcatagtgacgac

SEQ ID NO: 8 - DNA, 22 bp, Artificial Sequence
  fr Reverse Primer (reverse primer for PCR of E. coli phage fr)
aacggtcattcgcctccagcag

SEQ ID NO: 9 - DNA, 23 bp, Artificial Sequence
  fr Probe (probe for PCR of E. coli phage fr)
taggggatggtaacgacgaagca

SEQ ID NO: 10 - DNA, 22 bp, Artificial Sequence
  bact 100 Forward Primer (forward primer for PCR of bact 100)
gaaggtctcaaacatgatctgg

SEQ ID NO: 11 - DNA, 20 bp, Artificial Sequence
  bact 100 Reverse Primer (reverse primer for PCR of bact 100)
gggacgacatggagaaaatc

SEQ ID NO: 12 - DNA, 19 bp, Artificial Sequence
  bact 100 Probe (probe for PCR of bact 100)
ccccgtgctgctgaccgag

SEQ ID NO: 13 - DNA, 22 bp, Artificial Sequence
  H18 Forward Primer (forward primer for PCR of the human H18 gene)
gccgctagaggtgaaattcttg

SEQ ID NO: 14 - DNA, 21 bp, Artificial Sequence
  H18 Reverse Primer (reverse primer for PCR of the human H18 gene)
cattcttggcaaatgctttcg

SEQ ID NO: 15 - DNA, 21 bp, Artificial Sequence
  H18 Probe (probe for PCR of the human H18 gene)
accggcgcaagacggaccaga

(19) United States
(12) Patent Application Publication
(10) Pub. No.: US 2011/0189654 A1
(43) Pub. Date: Aug. 4, 2011

(54) DIAGNOSTIC REAGENT, CONTAINING BIOPARTICLES, METHOD FOR PRODUCTION THEREOF AND USE THEREOF AS INTERNAL STANDARD IN NUCLEIC ACID PREPARATION AND NUCLEIC ACID DETECTION METHODS

(75) Inventor: Ralf Himmelreich, Langenfeld (DE)

(73) Assignee: Qiagen GMBH, Hilden (DE)

(21) Appl. No.: 12/994,768

(22) PCT Filed: May 11, 2009
(86) PCT No.: PCT/EP2009/055646

§ 371 Date: Mar. 17, 2011

(30) Foreign Application Priority Data
May 27, 2008 (EP) 08156939.4

(51) Int. Cl. C12Q 1/68; C12Q 1/70; C12Q 1/02; C12M 1/34

(52) U.S. Cl. 435/5; 435/6.1; 435/6.12; 435/29; 435/287.2

(57) ABSTRACT

A diagnostic reagent in the form of a composition dimensionally stable under standard conditions, comprising bioparticles and also customary pharmaceutical excipients, wherein the bioparticles are selected from the group consisting of bacteria, viruses, fungi, protozoa, bacteriophages, yeasts, spores, parasites, plant cells, animal or human cells, gametes, plasmids, and viroids.

The present invention relates to a diagnostic reagent in the form of a composition which is dimensionally stable under standard conditions and which comprises at least one type of bioparticle selected from the group consisting of bacteria, viruses, fungi, protozoa, bacteriophages, yeasts, spores, parasites, plant parts, animal or human cells, gametes, plasmids, and viroids and also customary pharmaceutical excipients, to a method for production thereof, and to use thereof as an internal standard in methods for nucleic acid preparation and nucleic acid detection, more particularly in microfluidic devices. The invention further relates to a kit-of-parts, comprising the diagnostic reagent, and also to a method for detecting the bioparticles mentioned using the diagnostic reagent.

### BACKGROUND OF THE INVENTION

Detection of bioparticles, such as bacteria, viruses, fungi, protozoa, or the like, in samples of different origin is increasingly of great importance. For example, when selecting an appropriate treatment method or therapeutic agent for patients who have fallen ill because of infections, it is very important to determine which pathogenic agent is present. To date, in the case of suspected bacterial infections for example, samples, such as blood or urine samples for example, were often incubated for this purpose, and it was determined after a few days with the aid of lengthy tests with various antibiotics which pathogens are present and which therapeutic agents, if necessary, would be appropriate for the treatment. Often, valuable time is lost as a result. Detection of bioparticles in samples such as foodstuffs, drinking water, or the like for quality control is also increasingly important. Also the attacks with the anthrax pathogen Bacillus anthracis in the USA have made clear that very rapid and reliable analysis methods for microorganisms are urgently required.

Accordingly, there have been efforts for a long time to develop faster diagnostic methods which make the determination of pathogens possible after just a few minutes or hours.

One possibility consists in releasing the nucleic acids of the pathogens present in a sample, subsequently specifically amplifying these nucleic acids, and then detecting them. For this purpose, the following steps are required:
Withdrawal of material (e.g., blood, sputum, urine, feces, cerebrospinal fluid, and also swab samples from oral cavity, nasal cavity, genital tract)
Lysis (disruption of the cells)
Nucleic acid preparation
Nucleic acid amplification
Detection of the amplified nucleic acids

Disruption/lysis of cells is understood to mean the breaking open of cells, with nucleic acids being released from cells. Furthermore, proteins are released from the cell interior.

Cells can be disrupted with different levels of difficulty. Cultivated cell cultures can generally be simply lysed with agents which are comparatively gentle on the genetic information. In contrast, spores are comparatively difficult to disrupt. The agents required for the lysis of such cells not only break open the cells but also fragment much of the genetic information.

An example of cells which are simple to disrupt are white blood cells. These cells can be lysed comparatively gently, by the enzyme proteinase K in the presence of a detergent (e.g., sodium dodecyl sulfate or Triton X-100) for example.

With each cell disruption, nucleic acids are sheared, and fragmented via double-strand breaks in the genetic material. However, the genetic information is not fragmented to such an extent that the desired analyses would be compromised as a result.

The greater the extent of fragmentation of the nucleic acids, the more problematic it is to carry out the desired analyses. It is thus a goal in the disruption of cells to reduce double-strand breaks to a tolerable level.

When difficult-to-disrupt cells are to be lysed, this occurs, for example, by supplying heat. Difficult-to-disrupt cells are, for example, treated at 95° C. for 10 minutes. Typically, many bacteria are lysed with the aid of heat.

A further method is lysis by means of ultrasonication, which is used in the case of difficult-to-disrupt cells. However, ultrasonication leads to particularly extensive fragmentation of genetic information. Accordingly, ultrasonication is usually used only when other methods fail. Spores, for example, which are particularly resistant, are lysed with the aid of ultrasonication.

A third method for the lysis of difficult-to-disrupt cells is grinding by means of glass beads. With the aid of glass beads, bacteria and fungi are disrupted. Disruption by means of glass beads (also known as bead beating in professional circles) proceeds comparatively gently in comparison with ultrasonication and heat. However, in all three cases, the genetic information is fragmented to different extents.

When disrupting with glass beads, an apparatus for agitation or shaking, a Vortex® agitator for example, is generally used. One or more tubes composed of plastic are filled with an aqueous solution (which comprises a lysis buffer), the cell material, and glass beads, sealed, and placed into the apparatus for agitation or shaking, and shaken by means of the apparatus, for example for five minutes. Such a method is also referred to as vortexing with glass beads or as bead beating.

When the cell disruption is complete, the tube(s) is/are removed and centrifuged if necessary in order to separate the glass particles from the liquid, and the liquid content is added to a further tube. Therein, the lysed cell material is treated with further buffer solutions in order to isolate the nucleic acid.

Lysis by means of glass beads is described, for example, in the document DE 10 2004 032 888 B4.

After the lysis, the nucleic acids released can preferably be isolated from the mixture obtained, for example by solid-phase extraction on appropriate supports, such as silica, glass-fiber membranes for example, or by adsorption to magnetic particle surfaces. Subsequently, washing is carried out with appropriate solvents, more particularly with wash buffer solutions, and the nucleic acids are then eluted with appropriate elution buffers. These technologies are well known to persons skilled in the art, and appropriate adsorption materials, wash buffers, and elution buffers can be easily selected by means of tests.

The amplification of nucleic acids is usually carried out by polymerase chain reaction (PCR). Other amplification methods are, for example, ligase chain reaction (LCR), gap-filling LCR (Gap-LCR), nucleic acid sequence-based amplification (NASBA), and transcription-mediated amplification (TMA). These methods are well known in the prior art.

PCR is an enzymatic method for synthesizing specific nucleic acid sequences using two oligonucleotide primers (probes) which hybridize with opposing strands and flank an interesting section of a target nucleic acid. A series of repeated reaction steps which comprise template denaturation, primer annealing, and extension of the hybridized primers by DNA polymerase results in exponential accumulation of the specific target fragment, whose ends are defined by the 5′ ends of the primers. The PCR method uses repeated cycles of DNA synthesis to replicate the target nucleic acid. In the fundamental embodiment, PCR comprises the following steps:
Adding specific primers to a sample which is suspected of containing the target nucleic acid. The primers are chosen such that they bind to complementary DNA strands which are present in opposing strands of the DNA. The target nucleic acid is located within the primer binding sites and is a marker for the microorganism to be detected.
Adding the four deoxynucleoside triphosphates, magnesium sulfate-containing buffer, polymerase enzymes, and different additives and cosolvents; mixing with the sample and the primers.
Denaturing the sample by heating, separation of the DNA into two complementary strands.
Cooling down, resulting in binding (hybridization) of the primers to the respective binding sites on the complementary DNA strands.
Extending the primers on the DNA template by DNA polymerase.

These three steps of denaturation, hybridization, and extension are repeated from 40 to 50 times and result in an exponential amplification of the target sequences.

To detect the amplification products, suitable probes which are labeled, for example, with labels such as fluorescent labels (fluorophores) are added. These are oligonucleotides which are complementary to the target DNA. These probes hybridize with the target DNA and then make possible their subsequent detection, by fluorescence spectroscopy for example.

PCR technology is described extensively, for example, in PCR Technology: Principles and Applications for DNA Amplification Erlich, editor (1992); PCR Protocols: A Guide to Methods and Applications, Innis et al., editors (1990), R. K. Saiki et al., Science 230: 1350 (1985), and U.S. Pat. No. 4,683,202, whose disclosure is incorporated here fully.

The classic PCR reaction is an end-point PCR. As a result, it is possible to detect the presence or absence of a target DNA; however, the method gives no information about the starting concentration of the target DNA.

Recently, real-time PCR(RT-PCR; also called quantitative PCR (qPCR)) was developed. This makes possible the simultaneous tracking of multiple nucleic acid amplification reactions by PCR, with the accumulated data being used to quantitatively determine the starting concentration of a target nucleic acid sequence. Accordingly, this PCR reaction is also referred to as multiplex PCR. Real-time PCR is, for example, disclosed in EP-A-0 512 34, EP-A-0 640 828, EP-A-0 519 338 (F. Hoffmann-La Roche AG). For this purpose, commercial systems are offered, an example being TaqMan® (Roche Molecular Systems, Inc., Branchburg Township, N.J.). The method makes possible the rapid and precise quantification of the starting copy number over a very wide concentration range. It is suitable for many applications, such as gene expression studies, sequence and mutation analyses, checking virus titers, the detection of pathogenic or genetically modified organisms. A kit for real-time PCR having sequence-specific probes is available, for example, from Eppendorf AG, Hamburg (RealMasterMix Probe®) or from artus GmbH, Hamburg (RealArt® PCR Kits).

With TaqMan® PCR, the oligonucleotide probes are designed such that they bind to the target nucleic acid sequence upstream of the extension primer. Each oligonucleotide probe is labeled at the 5′ end with a reporter molecule, such as a fluorophore, and at the 3′ end with a reporter molecule quencher. The labeled probes are added together with the primers and the sample to the PCR reaction mix. After the denaturation, the reaction mix is cooled down, with the labeled probes binding preferably to the primers. Next, the mix is cooled down to the optimal temperature for primer binding and extension. During the course of the polymerization, when the DNA polymerase advances along the target nucleic acid strand from the 3′ end to the 5′ end, with nucleotides being attached to the growing primer, the primer encounters the 5′ ends of the labeled probes bound beforehand to the target nucleic acid strand. When the DNA polymerase encounters these labeled probes, it exercises its 5′-3′ endonuclease activity and degrades the labeled probes. Fluorophores and quenchers are released into the reaction mix. The TaqMan® assay is designed such that it does not detect reporter molecules which are present within a predetermined proximity of the quencher molecule. Therefore, in the PCR mix at the beginning, no fluorescence owing to the reporter molecule is detected because the fluorescence of the reporter molecule is quenched by the quencher molecule located in the spatial proximity. During the PCR, the 5′ fluorescently labeled probes are released and separated from the 3′ fluorescence quenchers. After the release, the fluorescent label is no longer quenched and can therefore be detected by fluorescence spectrometry or by other suitable means. Unbound probes present in the reaction mix do not interfere, since they remain quenched. Likewise, labeled probes bound unspecifically to nucleic acid sequences which have nothing to do with the target nucleic acid do not interfere, since they remain bound and therefore quenched. Therefore, every free reporter molecule which is detected in the reaction mix is directly proportional to the amount of the originally and specifically bound labeled probe, and therefore of the target nucleic acid. To detect different target nucleic acids in a sample, use is made of probes having different fluorescence reporter molecules which fluoresce at different wavelength ranges and therefore make possible the simultaneous detection of different target DNAs. Known, useful fluorescence reporters are, for example, 6-FAM, VIC, Bodipy-TMR, JOE, and HEX; known, useful quenchers are TAMRA, MBG, Black Hole Quencher 1 (BHQ1), Black Hole Quencher 2 (BHQ2), Black Hole Quencher 3 (BHQ3), BodyPi 564/570, and Cy5, or the like. A person skilled in the art appropriately selects the reporter and quencher, preferably such that a reciprocal overlapping of the fluorescence emissions (bleedthrough) is avoided. For example, the combination of 6-FAM and BHQ1 is particularly useful.

In addition to fluorescent labels, the labels used can also be radioactive labels, chemiluminescent labels, paramagnetic labels, enzymes and enzyme substrates.

Following the amplification, the nucleic acids are detected by fluorescence spectrometry, as described above, or, if other labels are used, by other corresponding detection methods.

The methods mentioned for nucleic acid preparation and nucleic acid amplification and also nucleic acid detection can be carried out on a laboratory scale, i.e., on a microliter or milliliter scale. Alternatively, microfluidic methods were recently developed.

Microfluidics is understood to mean handling and working with small liquid amounts which have a lower volume, by a few orders of magnitude, than normal liquid drops.

Devices for carrying out microfluidic methods which process a sample up to the desired measurement result are also referred to as lab-on-a-chip (LoC), i.e., a single-use consumable which is in the form of a laboratory on a chip having a similar size to a credit card and with which determinations by means of molecular biology are undertaken, examples being the determination of pathogens of an infection, foodstuff inspection, veterinary diagnostics, analysis of biological weapons, environmental analysis. For the analysis, only minimal personnel expenditure and no special education or training for operation are required. LoCs are inserted into an operator instrument which has the size of a video recorder and which then gives out the result. An LoC combines microfluidic functions for sample extraction and concentration of the analyte, for signal amplification and detection in a chip. The operation of the LoC and the operating unit is very simple, and the anaylsis is effected extremely quickly compared with conventional analysis methods (total processing time of 30-60 minutes compared with at least 6 hours).

A major problem in the steps for nucleic acid preparation and nucleic acid amplification, both on a laboratory scale and with microfluidic devices, is incorporating reliable checking of the method in order to avoid, more particularly, false-negative results. False negative means that the target nucleic acid is present in the sample because, for example, a certain pathogen is present, but is not detected owing to errors in the test. The lysis of the cells, the isolation of the nucleic acids and also the amplification may be prone to error. Especially PCR is sensitive to disadvantageous effects of inhibitors, many of which are often present in biological samples, such as heme and its metabolic products, acidic polysaccharides, detergents, and chaotropic salts for example. Likewise, the lysis of the cells does not always proceed reliably, more particularly in the case of difficult-to-lyse cells, as described above.

It has therefore already been proposed (WO 02/052041, WO 2005/04762) to add internal control substances to the PCR, which are designed such that there is confirmation of the amplification being carried out correctly. The internal standard may be added to the sample before the first processing step, for example before the purification or the lysis, or even before the PCR. Inter alia, there is produced a synthetic oligonucleotide construct which is different from the target region of the test detection system (internal control target). Appropriate selection of the internal control is very complex and difficult. Internal control standards known in the prior art are purified nucleic acids, protein-complexed nucleic acids (armored RNA from Ambion), with capsid for example, or inactive virus standards.

A problem with the purified nucleic acids is their low stability. Especially RNA is degraded in blood or other biological sample materials within a very short period of time. In addition, the use of nucleic acids does not make it possible to check whether the lysis, i.e., the sample disruption including nucleic acid release, has proceeded correctly; their use only makes it possible to check the amplification.

Armored RNA is distinctly more stable than “naked” nucleic acids, but is distinctly easier to lyse than intact cells and therefore not comparable with these cells and not well suited as a lysis control.

The inactive virus standards are essentially vaccines, and the chemicals present, more particularly formaldehyde, change the surface structure of the viruses, with respect to active forms, by chemical crosslinking. Therefore, the lysis behavior is not comparable with that of living viruses, and the inactive virus standards are not really suitable as a lysis control.

WO 03/02959 relates to products obtained by freeze-drying, comprising certain defined amounts of bioparticles. The application covers, according to information from the applicant in the last submission in the EP procedure of Aug. 22, 2006, the product BioBall®, which is freeze-dried beads which are used for quantitative, microbiological quality control. They are, to date, used in the food, pharmaceutical, water, and sewage industries, but can also be used for cosmetics, personal care, clinical laboratories, and research and academic institutions. Further constituents of the beads or use as internal controls in nucleic acid preparations is not disclosed. BioBalls® are available with different bacteria, for example, enterococci, E. coli, salmonellae. There is disclosed, inter alia, a set of substantially solid products which comprise a defined number of from 1 to 1000 microorganisms, selected from viruses, bacteria, yeasts, fungi, parasites, protozoa, cells, and mixtures thereof, with the solid products being able to be transferred between containers without loss of microorganisms and the microorganisms being able to be released in a liquid, and with the deviation of the number of the microorganisms from the defined number being not more than 10%. The purpose of these products is to provide an exactly defined, very low number of microorganisms for comparative samples in quality control.

WO 2004/055205 discloses the use of cells, viral particles, organelles, organelle-, viral-particle-, parasite- or bacterial-cell-containing cells which comprise at least one nucleic acid sequence which serves as an internal control target for nucleic acid assays, as an internal control. PCR is disclosed as being preferred. The cells mentioned are bacteria, viruses, fungi, parasites, eukaryotic cells, or plant cells, or even spores. It is described as being especially advantageous that both the nucleic acid preparation and the amplification can be checked by directly adding the cells to the test sample. Use can be made of genetically modified cells (i.e., those in which the control DNA sequence was incorporated by gene technology methods) or natural cells, such as spores of B. globigii for example, as control cells. Solid specimens are not disclosed, but suspensions which comprise the spores genetically modified by introduction of the control DNA.

WO 02/18635 discloses the use of nonviable particles which comprise an internal nucleic acid sequence as an internal control for nucleic-acid-based analyses. The particles are more particularly liposomes.

DE 21 40 747 discloses a method for mixing solid biological products with other solid materials by freeze-drying in the presence of a sugar.

WO 99/06594 discloses a method for producing stably encapsulated nucleic acids which are usable for monitoring all steps of nucleic acid preparations. The cells described are killed after introduction of the nucleic acids, for example by leaving to stand at room temperature or with chemical reagents.

EP 1 179 585 and WO 99/33559 disclose an integrated cartridge for the analysis of clinical or environmental liquids, more particularly for the detection of DNA. The system is commercially available from Cepheid under the name GeneXpert®. There are different internal controls for various assays, for example spores of Bacillus globigii for the assay for MRSA (methicillin-resistant Staphylococcus aureus), or armored RNA from Ambion for viral RNA.

The internal standards mentioned are all still not satisfactory. Firstly, most of them can be used only as a PCR control but not as a lysis control, since they are not intact microorganisms having a structure comparable to that of the microorganisms to be detected. Secondly, they are, in some cases, instable products which are not storable under standard conditions. For a simple procedure, the suspensions or solutions, some of which are known, are additionally disadvantageous because they are more laborious and inexact to dose and to add than, for example, solid specimens.

Both for methods on a laboratory scale and for microfluidic methods, there is an urgent need for reliable internal standards which make it possible to check that both the lysis and the amplification are carried out correctly. For methods on a laboratory scale, it should be considered that the internal standard can in principle be added before or after each individual step of the detection method, or that different standards can also be used for each step. With microfluidic methods, in contrast, the internal standard has to be added at the beginning of the method and be suitable for checking all steps, since adding is no longer possible in subsequent steps owing to the method being carried out in a closed system.

As explained above, there are to date still no satisfactory internal standards which make it possible to reliably check all steps of a detection method for bioparticles by analysis of nucleic acids thereof (i.e., preparation/lysis of the cells, extraction, amplification, and detection).

Food supplements which contain bacteria or their constituents and which are in tablet form are known.

EP-A 1 002 539 describes, for example, a tablet-formed foodstuff which comprises immunomodulatory degradation products of bacterial cells of Corynebacterium glutamicum, starch, and cellulose.

Furthermore, Derwent Publication, AN2008-055669-XP002501895 (JP-A 2008043206) discloses food supplement tablets which are composed of yeast cell wall fractions and live bifidobacterium. An object of the invention consists, in respect of the problems described, in providing a suitable internal standard for methods for nucleic acid preparation and nucleic acid detection.

### DISCLOSE OF THE INVENTION

The object is achieved by the diagnostic reagent according to claim 1, comprising at least one type of bioparticle selected from the group consisting of bacteria, viruses, fungi, protozoa, bacteriophages, yeasts, spores, parasites, plant cells, animal or human cells, gametes, plasmids, and viroids and also customary pharmaceutical excipients. Preferred embodiments are defined in claims 2 to 6. Likewise, there is claimed a production method for the reagent according to the invention and also its use as an internal standard in methods for nucleic acid preparation and nucleic acid detection and for quantitative determination of nucleic acids, a method for detecting the bioparticles mentioned using the diagnostic reagent, and also a kit-of-parts comprising the diagnostic reagent.

According to the invention, for the first time there has been obtained, surprisingly, a solid, stable product which is easily storable under standard conditions and at ambient temperature and which comprises bioparticles and can be used as an internal standard for methods for nucleic acid preparation and nucleic acid detection. It is obtainable in a very simple manner, with no laborious and expensive procedural steps such as freeze-drying being necessary.

### DESCRIPTION OF THE FIGURES

FIG. 1 is a photograph of an ethidium-bromide-stained agarose gel on which the sample eluates from example 2 were separated electrophoretically. Image A shows the nucleic acid preparation for blood and PBS with tablet A; image B shows the nucleic acid preparation for blood and PBS with tablet B.

FIG. 2 is a graph which is obtained in example 3 and which shows the result of the real-time PCR quantification of the genomic DNA of Corynebacterium glutamicum. The histogram shows the amount detected by PCR (pg) per reaction, with a sample size of N=4.

FIG. 3 is a graph which is obtained in example 4 and which shows the threshold cycles (Ct) of the quantitative PCR of individually prepared blood samples. For each sample, a Corynebacterium glutamicum-specific and a human-DNA-specific (β-actin gene) PCR was carried out.

FIG. 4 is a graph which shows the result of the quantitative PCR for detecting B. subtilis DNA from example 5 with different numbers of bacteria.

FIG. 5 is a graph of the amplification plot, said graph showing the result of the reverse-transcribed quantitative PCR with a specimen according to the invention in example 6.

### DETAILED DISCLOSURE OF THE INVENTION

Bioparticles are, according to the invention, understood to mean microorganisms, more particularly bacteria, viruses, fungi, protozoa, bacteriophages, yeasts, spores, parasites, plant cells, animal or human cells, gametes, plasmids, and viroids. The bioparticles comprise genetic material in the form of nucleic acids. Preferably, the specimen according to the invention comprises bacteria, viruses, and/or bacteriophages. Usually, the specimen comprises one type of microorganism, but it is also possible for multiple types to be present together, for example two or more types of bacteria. Also a combination of one bacterium type and one virus type or one bacterium type and one bacteriophage, for example, is possible. Further combinations are likewise contained within the scope of the invention. Especially useful are, according to the invention, bacteria and viruses which maintain their cell wall and morphology during the drying under standard conditions. These are, for example, the bacteria Corynebacterium glutamicum, Mycobacterium phlei, and Bacillus subtilis, the bacteriophages lambda, T7, fr, qβ; MS2 and M13. The bacterium E. coli is in principle also useful, but is less preferred for the specimen according to the invention because of its insufficient stability toward lysis (to easily lysable). C. glutamicum is especially useful because it is easily obtainable and difficult to lyse, i.e., is especially suitable for checking correct lysis. The phage fr is highly useful because it comprises an RNA genome and is only amplifiable via reverse transcription. Thus, it can be used advantageously for checking amplifications with reverse transcription.

Standard conditions and ambient temperature are, according to the invention, understood to mean room temperature and standard pressure, i.e., a temperature of about 20-25° C. and an air pressure of about 1 bar. This is further understood to include a relative humidity of 20-60%. Ambient air having a lower relative humidity can, according to the invention, also be used.

The reagent according to the invention is substantially solid. This means that the reagent is dimensionally stable under standard conditions, i.e., does not melt, disintegrate, or dissolve. It can, for example, be present in the form of pellets or tablets, or even in the form of clusters of the mixture of bioparticles and excipients, which are produced by spreading a certain amount on, for example, a film or pipetting into indentations and subsequent drying. In an alternative design, the reagent can be produced on a support nonwoven fabric, made of glass fiber for example. The punched-out products obtained from it represent the final reagent.

More particularly, the terms “substantially solid” and “dimensionally stable under standard conditions” are intended to signify that a liquid reagent in which bioparticles are present as a suspension in a solvent is not involved.

The reagent further comprises customary pharmaceutical excipients which are suitable for producing a solid preparation. More particularly, flow aids, binders, fillers, and/or disintegrants are used. These are well known to persons skilled in the art and are appropriately selected.

Useful fillers are, for example, starches, such as corn starch, potato starch, and wheat starch, lactose, Granulatum simplex (mixture of potato starch and lactose), microcrystalline cellulose (e.g., Avicel®) modified starches, modified celluloses, glucose, mannitol, sorbitol, and levulose.

Binders are important for increasing the strength of the solid specimen. Useful for this purpose are, for example, polyethylene glycols.

A useful flow regulator is, for example, Aerosil® (colloidal silica).

Useful disintegrants are customary disintegrants, such as, for example, starch, corn starch or potato starch for example, alginates, microcrystalline cellulose (Avicel®), holocellulose (purified lignin-free cellulose), sodium carboxymethyl cellulose, polyacrylic acid (Carbopol®), polyvinylpyrrolidone (Polyplasdone®, Kollidon®, Crospovidone®), crosslinked sodium carboxymethyl cellulose (Ac-Di-Sol®), sodium carboxymethyl starch (Primojel®, Explotab®), sodium carboxymethyl cellulose (Nymcel®).

Flow regulators, fillers, and disintegrants are appropriately selected. Preferably, use is made, according to the invention, of hydroxyethyl starch and/or carboxymethyl cellulose because sufficiently solid and stable specimens are obtained with them. Preferably, the binder used is polyethylene glycol 8000.

During the course of the production method, one or more buffers can be added to the reagent. Use is made of buffers which have stabilizing and lysing properties for microorganisms. Such buffers comprise detergents (e.g., SDS (sodium dodecyl sulfate), lauryl sarcosine, Tween 20 (polyoxyethylene sorbitan monolaurate), Triton X-100 (polyethylene glycol p-(1,1,3,3-tetramethylbutyl)phenyl ether), chelating agents (e.g., EDTA (ethylene glycol tetraacetic acid) or salts thereof, such as sodium EDTA (Titriplex)), EGTA (N-(2-hydroxyethyl)ethylenediamine), and/or pH buffer substances, such as Tris, HEPES (4-(2-hydroxyethyl)-1-piperazineethanesulfonic acid), MOPS (3-(N-morpholino)propanesulfonic acid), citrate, acetate, ammonium chloride). These constituents may be present on their own or as a mixture in the buffer used. The buffer, if necessary, is appropriately selected for the microorganisms by the person skilled in the art. For example, the buffer used can be, according to the invention, TE (Tris/Cl, pH 8, in a mixture with EDTA), or EDTA in combination with Tris/Cl, Tween 20, and Triton X-100, Buffer P (1.4% SDS, 50 mM Titriplex, 500 mM NaCl, 20 polyvinylpyrrolidone, 100 mM sodium acetate trihydrate, pH 5.4) or Buffer L (155 mM NH₄Cl, 10 mM KHCO₃) or Buffer B (50 mM EDTA, 50 mM Tris/Cl, pH 8.0, 0.5% Tween 20, 0.5% Triton X-100). At the end of the production of the reagents according to the invention, the solvents present in the buffers, more particularly water, are removed as much as possible by drying under standard conditions.

The amounts of the pharmaceutical excipients are appropriately selected. They can, if necessary, be determined by simple experiments. Typically, in a batch use is made, for example, of 3 g of binder for 1.5 ml of bacterial culture liquid or bacteriophage supernatant. The amount of the binder is likewise appropriately selected, or determined by experiments, and is, for example, 0.5 ml of a 20% solution of PEG 8000 for 1.5 ml of bacterial culture or bacteriophage supernatant. The amount of the buffer solution used is, for example, 1 ml of buffer solution for 1 ml of bacterial culture or bacteriophage supernatant.

The overnight bacterial culture is, according to the invention, obtained as follows: the bacterial cultures are grown as an overnight culture. For this purpose, 20 ml of an appropriate bacterial culture medium are pipetted into a sterile 100 ml Erlenmeyer flask and inoculated with 5-20 of bacterial suspension. The flask is loosely sealed with some aluminum foil and incubated for 10-14 h in a shaking incubator at an appropriate temperature and shaking setting. After the cultivation, the bacterial titer of a 1:10 diluted aliquot is determined by counting under the microscope with the aid of a modified Neubauer counting chamber.

Bacteriophage supernatants are produced by infection of host bacteria. For this purpose, 20 ml of an appropriate bacterial culture medium are pipetted into a sterile 100 ml Erlenmeyer flask and inoculated with 5-20 μl of bacterial suspension. The culture is incubated for 4 h in a shaking incubator at an appropriate temperature and shaking setting. Afterwards, an appropriate amount of bacteriophage solution is added, and cultivation proceeds for a further 10-12 h in the shaking incubator. The person skilled in the art generally uses an MOI (multiplicity of infection) of 10, i.e., the ratio of bacteria to bacteriophages is 1:10. After the cultivation, the bacteria are sedimented for 10 min at 8000 rpm in the centrifuge, and the supernatant is transferred to a fresh centrifuge tube and centrifuged again. The supernatant then comprises all free bacteriophages and can be frozen in aliquots. The phage titer (PFU: plaque forming units) is determined according to classical microbiology methods by plating out dilutions of the bacteriophage supernatant and indicator bacteria on agar plates. After the incubation of the agar plates, the number of PFU can be determined by counting the plaques in the bacterial lawn.

The appropriate number of bacteria and/or bacteriophages which is added during the production of the reagent will be decided by the person skilled in the art by reference to the final application. It is, for example, 5000. The minimum number is about 1000, since this value is the detection limit. Preferably, it is from about 10,000 to about 100,000.

The reagent according to the invention may, as required, comprise appropriate dyes in order to provide color coding. This means that solid reagents according to the invention are present differentially stained depending on the microorganism present therein and can thus be easily distinguished from one another by the user. For this purpose, it is worth considering dyes for histological stains, which are well known to persons skilled in the art and are appropriately selected. For example, use can be made of fuchsine, methylene blue, eosin, malachite green, gentian violet, and their derivatives. The dyes are added either as solids or in the form of a solution, for example, water and/or glycerol and/or customary alcohols, to the mixture for producing the reagent according to the invention. Examples which may be mentioned are gentian violet solution (in water), fuchsine solution (in ethanol), eosin solution (in water), methylene blue solution (in 95% ethanol), or malachite green solution (in water/glycerol with added acetic acid). Solutions of this kind are commercially available, for example from Fluka or Sigma-Aldrich.

As required, the reagent may also comprise antifoaming agents. These are appropriately selected from the known antifoaming agents.

Optionally, the reagent according to the invention may also comprise particles made of a hard material, such as glass, silicon carbide, or zirconium carbide. Also suitable for this purpose are magnetic silica particles or anion exchange resin particles. The diameter of these particles is preferably from 100 to 800 nm. This addition comes into consideration, more particularly, when the reagent comprises microorganisms which are very difficult to lyse, more particularly certain bacteria, such as Corynebacterium glutamicum, Mycobacterium phlei, yeast fungi; or spores of Bacillus anthracis. This embodiment is more particularly worth considering when the lysis, which is to be carried out with the addition of the as internal standard, is carried out by use of a Vortex® instrument or in a bead mill. As mentioned at the beginning, the hard particles support the lysis of the microorganisms in these methods through a mechanical action. The amount of the hard particles is appropriately selected and is, for example, from 10 to 20 g, preferably about 14 to 17 g, for 1.5 ml of bacterial culture liquid.

Depending on the application, use is made of glass particles having different diameters:

The reagent according to the invention may further optionally comprise one or more enzymes. These likewise support the lysis. For this purpose, proteases come into consideration, such as, for example, proteinase K, subtilisins; lipases and cell-wall-degrading enzymes, such as, for example, lysozyme, lyticase, or zymolase. The enzyme(s) and its/their amount(s) is/are appropriately selected.

The reagent may be produced by mixing the constituents, which are in some cases also present in a liquid form, with a spatula for example or any mixer and, if necessary, adding distilled water until a tough to viscous homogeneous mass is obtained. The microorganisms to be incorporated are, in this embodiment, added to the rest of the constituents in the form of a bacterial culture liquid or a suspension of microorganisms, such as, for example, a phage supernatant. The sequence of adding is only important when the reagent is intended to additionally comprise hard particles. Prolonged stirring can lyse the bacteria in this case as a result of grinding effects; therefore, the suspension of microorganisms in this case is added as the very last ingredient and also mixed with the rest of the constituents only very carefully. Otherwise, the sequence of adding and mixing the individual constituents is not important.

The viscous mass obtained is, for example, transferred to a syringe and applied in portions onto an appropriate film, such as, for example, Parafilm or polyethylene film or Teflon film, with a blunt cannula (e.g., as small beads (diameter of 1-2 mm for example)) and then dried under standard conditions. The portioning can also be carried out with another appropriate customary portioning device. Alternatively, the viscous mass may also be filled into appropriate molds, such as, for example, tablet blisters, and then dried under standard conditions. In addition, a solid specimen may be obtained by applying the viscous mass on a support nonwoven fabric, made of glass fiber for example, drying, and punching out the desired shapes. Usually, the drying is achieved over a few hours, overnight for example, preferably in the fume cupboard. Surprisingly, neither cooling nor working under an inert gas is required.

In a further embodiment, all constituents of the solid reagent with the exception of the microorganisms are mixed, portioned, and dried as above. Then, a solution or suspension of the microorganisms is subsequently applied onto the solid products obtained, by use of a pipet for example, and drying is then carried out again under the abovementioned conditions. As a result of this, the solid product is impregnated as it were with the microorganisms. Surprisingly, a stable, solid reagent comprising microorganisms is obtained in this manner, too. This approach is especially suitable for both bacteria and phages. It offers the advantage that the amounts of the microorganisms applied onto each tablet can be exactly defined by titer determination, according to a customary method, and subsequent limiting dilution.

The solid reagents according to the invention which are obtained are stable for multiple weeks under standard conditions. This means that, on resuspension of the reagent, stored under standard conditions, in water or aqueous buffer solution after multiple weeks, a sufficient number of live microorganisms is still present, enabling use as an internal standard in methods for nucleic acid preparation and nucleic acid detection.

This is very surprising, since it has been assumed to date that stable microorganism specimens of this kind could only be obtained by lyophilization and would only be stable with the addition of sugars, such as, for example, trehalose or mannitol.

The solid reagent according to the invention is easy to produce, with laborious and expensive methods, such as lyophilization, not being required and with additives, such as the sugars mentioned, not being necessary.

The reagent is easy to handle, since the user can simply add it to the sample to be analyzed, such as, for example, blood, cerebrospinal fluid, or the like. As a result, contact with the microorganisms present in the reagent and losses of material and also contamination of instruments and laboratory surroundings, which inevitably occur when using suspensions or liquids, are avoided at the same time.

The handling of the reagent according to the invention becomes especially simple when it is already present in microfluidic devices or LoCs, and so the user no longer comes directly into contact with the reagent and the possibility of forgetting the internal standard does not exist in the first place. Therefore, there is also provided according to the invention a kit-of-parts, for example a microfluidic device, a detection cartridge, or an LoC, said kit-of-parts comprising the diagnostic reagent according to the invention.

The reagents according to the invention are used as internal standards in methods for nucleic acid preparation and nucleic acid detection. This means that they are, in customary methods for nucleic acid preparation and for nucleic acid detection, added before or during the first reaction step or even not until after the preparation of the detection mix.

Particularly preferably, their use can take place in microfluidic preparation and detection methods, where they can be added to the sample to be prepared before the application on the microfluidic device. Alternatively, it is also possible to provide the reagent according to the invention in the microfluidic device such that, on the application of the sample to be prepared, it is suspended in this sample and then further prepared together with the sample, i.e., as a constituent of an LoC or a detection cartridge.

The solid reagent according to the invention can be used as an internal standard for any customary disruption or lysis method, as described above. The sample is, for this purpose, mixed together with the reagent according to the invention and an appropriate lysis buffer. Subsequently, the mixture is treated by means of heat, ultrasonication, or bead beating. The lysis buffer is, for example, Buffer AL (QIAGEN GmbH, Hilden, Germany). Further lysis buffers are known to persons skilled in the art and are appropriately selected for the microorganism to be lysed, as are the appropriate lysis conditions. It is also possible to use multiple different lysis buffers. For example, a lysis buffer which can be used is PBS buffer (phosphate-buffered saline) which comprises proteinase K and lysozyme. If necessary, there is an initial incubation followed by, if necessary, the addition of a further buffer, such as, for example, Buffer G (GITC (guanidinium thiocyanate)/Nonidet® (nonylphenylpolyethylene glycol)) and reincubation. The appropriate volume of the buffer(s) has to be determined as a function of the sample material and is, for example, between 50 μl and 1 ml depending on the sample material. The temperature and time for the incubation are each appropriately selected. Since most lysis methods are carried out by means of using enzymes, the chosen temperature is also a function of the activity of the enzymes. Zymolase, lyticase, and lysozyme are generally used in the temperature range of 20° C. to 37° C. Proteases, proteinase K or subtilisin for example, are generally incubated at 50-60° C. The duration of the incubation is generally from 5 to 20 minutes, preferably about 10 minutes. The incubation can, for example, be carried out by shaking in a thermal mixer, at about 1400 rpm for example, or even on a Vortex® instrument (maximum setting), with the addition of glass beads if necessary, and the specimen itself according to the invention can comprise particles made of a hard material and/or enzymes to support the lysis.

Following the lysis, the nucleic acids released by the lysis are isolated by means of any purification method known in the prior art. For this purpose, an appropriate solvent, ethanol for example, can be added to the mixture obtained. The nucleic acids present in the lysate are generally purified by adsorption.

The purification methods by means of adsorption make use of the selective binding of the constituents of the content of the lysate to or on a solid support or carrier material (binding), the removal of undesired constituents from the solid support or carrier material (washing), and the dissolving of the desired constituent (eluting).

In order to allow a desired absorbing and desorbing during the purification of the biomolecules, there have been developed special filter elements which are, for example, formed from silica gel and which, firstly, are porous or matrix-like in order to allow a liquid to pass through the filter element and which, secondly, have a surface to which the biomolecules bind in a specific or unspecific process. In other purification methods, biomolecules are retained on filter elements simply through the size-exclusion effect. When a liquid which comprises a biomolecule, such as a nucleic acid for example, passes through the filter element, the biomolecules or a portion thereof remain in any case in the filter element, whereas the rest passes through the filter element. Furthermore, in order to recover the biomolecule from the filter element, an elution liquid, nuclease-free water for example, is directed onto the filter element to desorb the biomolecule. In this way, the desired biomolecule is detached from the filter element (eluted) and collected in a vessel. Such filter elements are often designed as membranes which are either arranged in individual vessels which have an inlet and an outlet or which are arranged in multiwell plates. The filter elements are processed either with centrifuges (spin format) or with apparatuses based on vacuum technology. Individual vessels which have an inlet and an outlet and which have a membrane and which can be used in a centrifuge are also known as columns, spin columns, single spin columns, filter vessels, or chromatography columns.

A further embodiment of the adsorptive media for nucleic acid isolation is represented by magnetic silica particles. In principle, the control reagents proposed in the invention are also compatible with these methods of nucleic acid isolation.

QIAGEN GmbH in Hilden, Germany provides a broad spectrum of purification protocols and of products required therefor for different biomolecules from a multiplicity of biological samples, based on the basic principle of the bind-wash-elute protocol. For example, the product QIAGEN QIAprep Spin Miniprep Kit is commercially available. For this purpose, different filter materials and filter devices are used, as described in WO 03/040364 or U.S. Pat. No. 6,277,648 for example.

To process a sample, it is thus known to use a vessel which is open at the top and closable. At the base of the vessel, there is located a membrane as a filter element. Below the membrane, there is located an opening which is in the form of a nozzle and which is connected with, for example, a piece of tubing. Via this piece of tubing, liquid can be sucked off from the vessel.

For processing, the biological sample is added at the top of the vessel. Subsequently, each intended solution is added, i.e., for example, a lysis buffer first of all to disrupt the sample. When the sample has been disrupted, the lysis buffer is sucked out of the vessel in a downward direction. The lysate comprises chemical/physical conditions which cause a binding of the nucleic acid to the carrier material. The disrupted sample (lysate) is then washed and eluted.

After the nucleic acid of the disrupted sample has been bound or adsorbed to the membrane, wash buffers are added to the vessel from above and sucked off or centrifuged from the vessel in a downward direction. The wash buffer maintains this binding while it, at the same time, washes undesired cell constituents from the membrane.

Wash buffers generally comprise ethanol. Ethanol causes a binding of nucleic acid under certain conditions to a membrane acting as a filter element. A membrane serving as a filter has a certain dead volume of, for example, 40 μl. It is therefore not possible to prevent a corresponding amount of ethanol from always remaining in the filter. Ethanol, firstly, makes an undesired contribution to the binding. Secondly, however, ethanol can also distort the result of a subsequent analysis. In order to be able to extract the nucleic acid from the vessel in a downward direction and in order to avoid flawed analysis results, ethanol is first removed, for example, by centrifuging the vessel. The ethanol thus escapes from the membrane and can be extracted in a downward direction. Following this, the membrane is sufficiently free from ethanol. By means of vacuum and supplying heat, ethanol can be alternatively removed from the membrane.

In order to detach the nucleic acid from the membrane, it is eluted. This routinely takes place with water or an aqueous pH-stabilized solution having a weak salt content. Following the elution, the nucleic acid can be extracted from the vessel, through the membrane, in a downward direction.

Alternatively, adsorption to magnetic particles and subsequent washing and elution can also be carried out.

The appropriate adsorption materials, the wash buffers, and the elution buffers are known to persons skilled in the art and are appropriately selected according to the invention.

Advantageously, adsorbing/washing/eluting when preparing DNA can be carried out according to, for example, the QIAamp DNA Micro Handbook (www1.qiagen.com/HB/QIAampDNAMicro) on a QIAamp MinElute spin column, which is provided by QIAGEN. The binding is followed by centrifugation and then washing with one or, if necessary, more appropriate buffers, for example with Buffer AW1 (guanidinium hydrochloride, ethanol), Buffer AW2 (NaCl, Tris/Cl, pH 7.5, ethanol), usually with recentrifugation after each wash step in order to remove the wash buffers. For the subsequent elution of the nucleic acids, the buffer TE (10 mM Tris/Cl, pH 8.0, 1 mM EDTA) can be used for example. Usually, the elution is likewise followed by centrifugation in order to remove the nucleic acids completely from the membrane.

Alternatively, an RNeasy spin column for example from QIAGEN can also be used for the RNA preparation and handled according to the RNeasy Mini Handbook (www1.giagen.com/HB/RNeasyMini). In this case, use can be made of, for example, the wash buffers Buffer RW1 (guanidinium isocyanate, ethanol) and RPE (Tris/Cl, pH 7.5, ethanol) and, for the elution, RNase-free water, with elution likewise being advantageously carried out after each wash/elution step.

When carrying out the preparation method and detection method in microfluidic devices, the wash buffers preferably used are Buffer AW1 or Buffer RW1, and Buffer AW2 or Buffer RPE, and the elution buffer preferably used is RNase-free water. The centrifugation after each wash or elution step is omitted.

Any customary method is likewise suitable as a nucleic acid detection method for the application of the reagent according to the invention. Preference is given to using the specimen according to the invention in PCR detection methods, more preferably in duplex real-time PCR methods as described at the beginning. The explanation of PCR can be found in the explanation given at the beginning.

The choice of the microorganism present in the reagent according to the invention determines which type of nucleic acid can be detected in the sample to be analyzed. When using bacteria, both RNA and DNA can be detected, preferably DNA is detected. Detecting RNA from bacteria is possible in principle, but is not meaningful, since not only the RNA specifically but also the DNA is amplified. When using bacteriophages and viruses, the nucleic acid type of the genome in the virus particle determines whether RNA or DNA is detected in the sample. When using protozoa or yeast fungi as a control microorganism, not only chromosomal DNA thereof can be detected, but also a selective amplification of RNA is possible, since these organisms have mechanisms for mRNA splicing.

To carry out the PCR, all current reagents, and kits available therefor, are usable. The primers, deoxynucleoside triphosphate, polymerase, the additives, cosolvents, and labeling probes are, as described at the beginning, known to the person skilled in the art and are appropriately selected. For example, for a TaqMan® analysis, use can be made, according to the invention, of appropriate PCR reagent kits from QIAGEN, such as the QuantiTect Probe PCR Master Mix, the QuantiTect Probe RT-PCR Master Mix, and the QuantiTect RT Mix.

The primers used can be, for example, “fr Forward” (CTTCTGATCCGCATAGTGACGAC) (SEQ ID NO: 7), “fr Reverse” (AACGGTCATTCGCCTCCAGCAG) (SEQ ID NO: 8), and “fr Probe” (‘6-FAM’-TAGGGGATGGTAACGACGAAGCA-‘BHQ1a’) (SEQ ID NO: 9), especially advantageous in this combination when the microorganism used is the phage fr.

More particularly when using the microorganism Corynebacterium glutamicum, the combination of CG Forward (AAGCTCCAGCCACCCAAACTAC) (SEQ ID NO: 1), CG Reverse (CTACCAACCACTAATGCGTCATC) (SEQ ID NO: 2), and CG Probe (‘6-FAM’-ATCGCCTTCCAGACGCTCAACG-‘BHQ1a’) (SEQ ID NO: 3) is used advantageously.

When Bacillus subtilis is used as a control microorganism, this can be detected with the following primer combination: BS Forward (ACATCTTACCGCAACTACGACCAT) (SEQ ID NO: 4), BS Reverse (TAGCATAGTCTTTGTCCCACCGTA) (SEQ ID NO: 5), and BS Probe (‘6-FAM’-GGAGGCGATCTATGTCTTGTCCA-‘BHQ1a’) (SEQ ID NO: 6).

The primers can be produced synthetically with methods known to persons skilled in the art or are commercially available from Operon or MWG Biotech.

Labels which can be used are all labels which are known to persons skilled in the art. More particularly, fluorescent labels are used for detecting the control microorganisms, especially advantageous being the combination of 6-FAM at the 5′ end of the oligonucleotide and Black Hole Quencher (BHQ) at the 3′ end. For detecting the microorganisms to be detected in the sample, appropriate labels which differ from those mentioned, HEX for example, are likewise selected. The person skilled in the art will, as required, also use other fluorophores.

The solvent usually used for the PCR reagents and primers is water.

To the PCR, there are added in addition template aliquots which are in the form of the eluate and which comprise the nucleic acids to be amplified.

The temperature cycles of the PCR are appropriately selected by the person skilled in the art depending on the underlying starting nucleic acid and can, for example, be 15 min at 95° C. and 40×[15 seconds at 95° C., 1 minute at 60° C.] or, for an RNA-based amplification, be 30 minutes at 50° C. (reverse transcription), 15 minutes at 95° C., and 40×[15 seconds at 95° C. and 1 minute at 60° C.]. The checking of the temperature and the warming and cooling are carried out in the usual manner.

The amplification products can, depending on the labels used, be detected in different ways as described at the beginning. This is known to the person skilled in the art. When using fluorescent labels, the detection is carried out by means of fluorescence spectrometry. In customary PCR methods, up to 6 different fluorophores can be detected simultaneously (6-channel multiplexing). The number of PCR cycles from which a distinct rise in the fluorescence of the specific label occurs (Ct, threshold cycle) is then determined.

When, during this detection, amplification products of the nucleic acids both from the control microorganism and from the sample to be analyzed are observed, it can be assumed that both the preparation and the amplification have proceeded correctly for the two nucleic acids and that the detection of the microorganism to be analyzed is positive.

When only amplification products of nucleic acids of the microorganism to be detected, but not of the control microorganism, are observed or when no amplification products are observed at all, the preparation and/or the amplification was not correct and has to be repeated.

When only amplification products of the nucleic acids of the control microorganism are observed, but no amplification products of the microorganism to be detected are observed, the preparation and amplification were correct and the test result is negative.

Kits-of-parts according to the invention include the diagnostic reagent according to the invention and also all solvents and reagents required for carrying out the desired reactions for nucleic acid preparation and/or nucleic acid detection, such as primers which are mentioned above and appropriately selected by the person skilled in the art.

The following examples describe the invention in detail.

The microorganisms used in the examples are commercially available from the American Type Culture Collection, Manassas, Va., USA. They include Bacillus subtilis ATCC 23857, Corynebacterium glutamicum ATCC 13032, and E. coli Phage fr ATCC 15767-B1.

The terms “standard deviation”, “mean”, and “coefficient of variation (CV)” are used below as is customary and determined with customary statistical methods. For diagnostic samples, a CV should be less than 8%.

### Example 1

### Production of Tablets

a) Tablet A (Corynebacterium glutamicum)

3 g of hydroxyethyl starch, 0.5 ml of 20% PEG 8000, 1.5 ml of TE (10 mM Tris-Cl, pH 8.0, 1 mN EDTA), 1.5 ml of an overnight culture of Corynebacterium glutamicum (obtained as described above) in 2YT medium (yeast extract tryptone medium), and a few crumbs of fuchsine (Fluka) are added to a single-use weighing dish and mixed to a homogeneous mass with a spatula. The mass is transferred to a syringe with the spatula and portioned onto Parafilm film as small beads (diameter of 1-2 mm) via a blunt cannula. Subsequently, the tablets obtained are dried overnight in the fume cupboard. There are obtained in this way, depending on the aliquots used, 50 to 200 tablets.

b) Tablet B (Corynebacterium glutamicum)

1 g of hydroxyethyl starch, 500 of 20% PEG 8000, 500 of an overnight culture of Corynebacterium glutamicum in 2YT medium (obtained as described above), 1 small spatula tip of methylene blue, 750 of Buffer P (1.4% SDS, 50 mM Titriplex, 500 mM NaCl, 20 polyvinylpyrrolidone, 100 mM sodium acetate trihydrate, pH 5.4), 500 of Buffer L (155 mM NH₄Cl, 10 mM KHCO₃, and 50 mg of carboxymethyl cellulose are, in the same way as for Tablet A, mixed, portioned on film, and dried overnight.

### c) Tablet C (Phage fr)

3 g of hydroxyethyl starch, 150 mg of carboxymethyl cellulose, 1 ml of 20% PEG 8000, 1 ml of Buffer B (50 mM EDTA, 50 mM Tris/Cl, pH 8.0, 0.5% Tween 20, 0.50 Triton X-100), 1 ml of double-distilled water, 1 small spatula tip of eosin, and 1 ml of phage supernatant of the phage fr (obtained as described above) are, in the same way as for Tablet A, mixed, portioned, and dried overnight.

### d) Tablet D (Phage fr)

The tablet was produced in the same way as for Tablet C, but with the exception that 1 ml of phage supernatant solution was replaced by 1 ml of double-distilled water. After the drying, 4 μl each of phage supernatant solution (obtained as described above) were pipetted onto each dried tablet, and redrying was carried out overnight in the fume cupboard.

e) Tablet E (Bacteria with Glass Beads)

0.75 g of hydroxyethyl starch, 150 mg of polyvinyl alcohol (30,000-70,000), 150 mg of polyvinylpyrrolidone (K15, MW ˜10,000), and 15.9 g of glass beads (425-600 μm) (Sigma-Aldrich G8772-500G) were weighed in a weighing dish. After addition of 1.5 ml of Buffer B (50 mM EDTA, 50 mM Tris/Cl, pH 8.0, 0.5% Tween 20, 0.50 Triton X-100), 1.5 ml of 20% PEG 8000, 20 μl of malachite green solution (Fluka), and also 90 μl of silicone-based antifoaming agent from Wacker Chemie, a homogeneous suspension is produced by thorough stirring. Then, 1.5 ml of bacterial culture liquid (obtained as described above) is stirred very carefully into this mass. Aliquots of this mass are then dried on an appropriate base.

f) Tablet F (Bacteria with Glass Beads)

1.5 g of hydroxyethyl starch, 50 mg of CMC (carboxymethyl cellulose), and 8 g of glass beads (100 μm) (Sigma-Aldrich G4649-500G) are weighed in a weighing dish. After addition of 0.5 ml of Buffer B (50 mM EDTA, 50 mM Tris/Cl, pH 8.0, 0.5% Tween 20, 0.50 Triton X-100), 0.5 ml of 20% PEG 8000, 1.25 ml of double-distilled water, and 1 drop of gentian violet solution (Fluka), a homogeneous suspension is created by careful stirring. Then, 0.75 ml of bacterial culture liquid (obtained as described above) is stirred very carefully into this mass. Aliquots of this mass are then dried on an appropriate base.

### Example 2

### Nucleic Acid Preparation and Nucleic Acid Amplification

### a) Lysis/preparation

One each of Tablet A was added to a 2 ml tube, and 200 μl of blood (example 2a) or 200 μl of PBS buffer (example 2b) were added. To each tube, 20 μl of QIAGEN proteinase K solution and 40 μl of lysozyme solution (20 mg/ml in water in each case) were added in each case. The incubation was carried out over 10 minutes at 56° C. on an Eppendorf Thermomixer at a shaking speed of 1400 rpm. Subsequently, 200 μl of Buffer G (3 M GITC (guanidinium thiocyanate), 20% Nonidet® P40) were added, and reincubation was carried out over 10 minutes at 56° C. on an Eppendorf Thermomixer at a shaking speed of 1400 rpm. Then, 200 μl of ethanol were added and mixed well with the tube content.

### b) Purification

The following steps were then carried out according to the QIAamp DNA Micro Handbook with each of the two samples:
Binding: the entire mixture was added to a QIAamp MinElute spin column and centrifuged for 1 minute at 6000×g
Washing: wash with 500 μl of Buffer AW1, centrifuge for 1 minute at 6000×g, add 500 μl of Buffer AW2, centrifuge for 3 minutes at maximum g-force
Drying: dry spin (drying step for the spin column membrane in the centrifuge in the absence of any wash buffer residues) for 1 minute at maximum g-force in order to remove all the solvent
Elution: elute with 80 μl of Buffer TE (10 mM Tris/Cl, pH 8.0, 1 mM EDTA), centrifuge for 1 minute at 6000×g

The same method was carried out for Tablet B.

### c) Quantitation by UV Quantification and Agarose Gel Electrophoresis

The quantitation of the nucleic acids was carried out for all eluates obtained (Tablets A and B, each in blood and PBS) by means of measurement of the optical density at 260 nm on a spectrophotometer. The optical density was determined in each case with a light wavelength of 260 nm. The optical density is a measure of the amount of the DNA present in the eluate. 1 OD corresponds to 50 mg/ml double-stranded DNA. From the optical density, the concentration of the DNA present in the eluate of the sample can thus be calculated (when using an appropriate dilution).

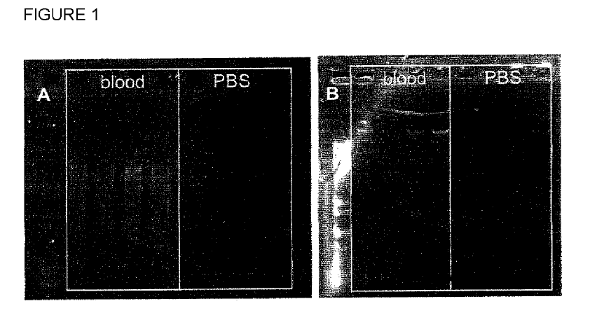

After the preparation according to the protocols described, the nucleic acids were separated electrophoretically on an agarose gel. A solution of 0.7% w/v agarose was heated to melt the agarose particles, and it was, after the homogenization, allowed to solidify in the form of a gel. The gel contained wells into which the solution containing nucleic acids was pipetted and electrophoretically separated. In this case, 15 μl of the eluate after nucleic acid preparation were applied, with FIG. 1 showing the results of Tablet A in image A and the results of Tablet B in image B.

After the separation, the nucleic acid fragments were stained in a water bath containing ethidium bromide, illuminated on a UV light box, and photographed by means of a photo documentation system. The genomic DNA can be seen as bright bands in the upper third of the lefthand side (blood) of FIG. 1A and in the lower half of the lefthand side (blood) of FIG. 1A.

It is clearly evident that only for the blood samples is genomic DNA (from the white blood cells) visible in the gel, whereas for the PBS samples, no DNA is present according to the gel. This shows that the amount of Corynebacterium glutamicum used in specimens A and B according to the invention is too low to be detected in the agarose gel electrophoresis.

The results of the quantification of the optical density of the eluates of the amounts of nucleic acids obtained in example 2 (before the PCR) are shown in table 1 below:

TABLE 1

| | |
|---|---|
| Tablet A | Tablet B |

| | Conc. | Yield | Conc. | Yield |
|---|---|---|---|---|
| Sample | (ng/μl) | (μg) | (ng/μl) | (μg) |
| Blood | 23.5 | 1.88 | 14.7 | 1.18 |
| Blood | 30.4 | 2.44 | 6.4 | 0.51 |
| Blood | 34.7 | 2.78 | 22.4 | 1.79 |
| Blood | 25.3 | 2.03 | 25.9 | 2.07 |
| PBS | 4.3 | 0.35 | 5.9 | 0.47 |
| PBS | 5.5 | 0.44 | 9.7 | 0.78 |
| PBS | 6.2 | 0.50 | 14.1 | 1.13 |
| PBS | 6.4 | 0.51 | 10.8 | 0.86 |

In the blood samples, about 20-30 ng/μl DNA (from white blood cells) were found in each case, whereas in the PBS samples, only about 5-6 ng/μl DNA, which originated from the C. glutamicum used in the specimen, were present. Thus, while the gel electrophoresis is not sensitive enough to detect the low amounts of control microorganism, the sensitivity of the photometric determination is sufficient for this purpose.

### Example 3

### Quantitative PCR (Specific for C. glutamicum)

The real-time PCR specific for C. glutamicum was carried out with 10.0 μl of QIAGEN QuantiTect Probe PCR Master Mix, 0.1 μl of CG Forward (SEQ ID NO: 1) (100 μM), 0.10 μl of CG Reverse (SEQ ID NO: 2) (100 μM), 0.05 μl of CG Probe (SEQ ID NO: 3) (100 μM), 7.75 μl of double-distilled water, and 2.0 μl of template aliquots of the eluates obtained in example 2 with blood and PBS samples with, in each case, tablets A and B. The temperature cycles were 15 minutes at 95° C. and 40×(15 seconds at 95° C., 1 minute at 60° C.). The amplification was carried out in each case with eluates of 4 individual nucleic acid preparations.

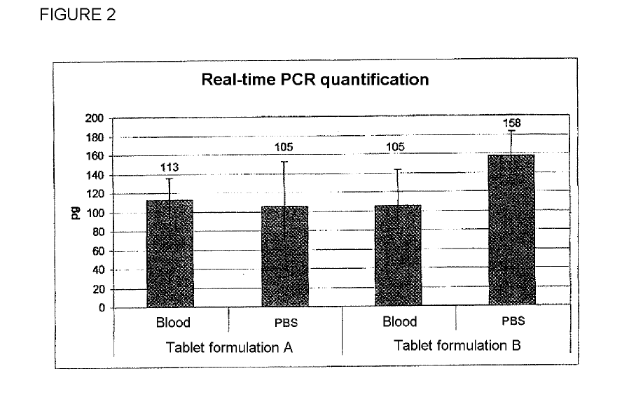

The results are shown in FIG. 2. The histogram shows the amount of C. glutamicum DNA detected by means of the PCR (in pg) per reaction with a sample size of 4. In each case, the Ct values in the RT-PCR were determined by means of fluorescence spectroscopy. With the aid of a calibration curve, the respective starting concentration of the DNA in the respective eluate was determined. The values of the 4 different batches and preparations lie within one standard deviation. The amount of C. glutamicum is about the same in all samples and batches. This shows that the specimens according to the invention can be used reproducibly for checking both the preparation of nucleic acids and the PCR.

### Example 4

### Duplex qPCR

Specimens of C. glutamicum were produced according to the formula for Tablet B (example 1b). In three independent determinations, 1 specimen each was given 200 it of whole blood. According to the protocol of example 2, the nucleic acids were prepared, and, with 2 it of eluate in each case, a quantitative duplex RT-PCR was carried out with the primers specific for C. glutamicum, CG Forward (SEQ ID NO: 1), CG Reverse (SEQ ID NO: 2) and the probe CG Probe (SEQ ID NO: 3), and the primers specific for the human β-actin gene, bact100 Forward (GAAGGTCTCAAACATGATCTGG) (SEQ ID NO: 10), bact100 Reverse (GGGACGACATGGAGAAAATC) (SEQ ID NO: 11) and the probe bact100 Probe (CCCCGTGCTGCTGACCGAG) (SEQ ID NO: 12), with the fluorophores HEX and BHQ. The reaction conditions were chosen according to example 3, with the exception that the primers were replaced according to the target DNA as mentioned above.

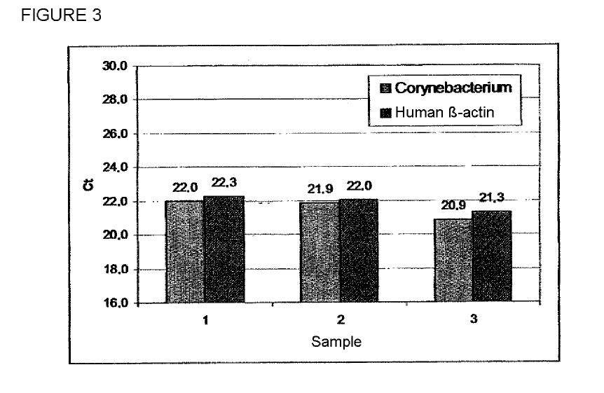

The threshold cycles (Ct) of the quantitative PCRs of the individually prepared blood samples were compared. The results are shown in FIG. 3. It becomes apparent that the Cts are about the same for both C. glutamicum and β-actin. This means that the specimen according to the invention reliably enables the checking of the reaction conditions in preparation and amplification and does not interfere with the preparation and amplification of the target gene human β-actin.

### Example 5

### Posttreatment of Tablets with Different Cell Numbers of B. subtilis

Tablets were produced in the same way as for Tablet C (see example 1c), but with the exception that 1 ml of phage supernatant was replaced by 1 ml of double-distilled water and dried overnight under standard conditions. Subsequently, different amounts of B. subtilis, each in 10 μl of culture medium, were pipetted onto the dried tablets. The specimens were then redried overnight under standard conditions.

The cell numbers used were:

a) 5×10⁵
b) 1×10⁵
c) 5×10⁴
d) 1×10⁴
e) 5×10³
f) 1×10³

The nucleic acid preparation was carried out in each case in quadruplicate, with 200 μl of blood in each case and one of tablets a) to f) in each case and with the addition of 20 μl of proteinase K (QIAGEN). This mixture was, in each case, shaken in the Thermomixer for 10 minutes at 56° C. After the addition, in each case, of 200 μl of Buffer G (3 M GITC, 20% NP-40), shaking was carried out again in the Thermomixer for 10 minutes at 56° C. The purification was carried out as described in example 2, by means of a QIAamp spin column.

The quantitative PCR was carried out according to example 3 with the primers specific for B. subtilis, BS Forward (SEQ ID NO: 4), BS Reverse (SEQ ID NO: 5) and the probe BS Probe (SEQ ID NO: 6), and also with the primers specific for the human gene for H18s, H18 Forward (SEQ ID NO: 13), H18 Reverse (SEQ ID NO: 14) and the probe H18 Probe (SEQ ID NO: 15), and the fluorophores HEX and BHQ.

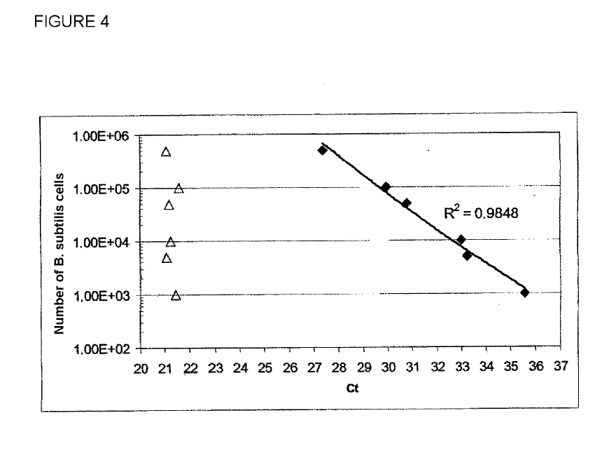

The result is shown in FIG. 4. There are shown averaged Cts of the 4 replicates with tablets a) to f) as a function of the cell numbers used (filled diamond). As is apparent from the regression lines and the specified coefficient of determination, there is a linear correlation between the cell number used and the Ct. The open triangles show the associated Ct values of the human H18 gene. Values at a similar height belong together to a particular sample. The values of the H18 qPCRs together have an average of 21.2 and a coefficient of variation (CV) of 0.96%.

It becomes apparent, from the regression lines in their coefficient of determination (R²) and also the low CV, that the specimens according to the invention can also be produced by posttreatment of the tablets with the microorganisms and are very highly suitable for methods for nucleic acid preparation and nucleic acid amplification as an internal standard. A reliable detection limit for the specimens according to the invention is 1000 cells per tablet.

### Example 6

### Reverse Transcription

With the addition of Tablet C (see example 1c), a plasma sample was prepared and amplified as follows using the QIAamp Virus Kit (QIAGEN GmbH, Hilden, Germany):

One Tablet C was mixed in the Thermomixer for 2 minutes at 1400 rpm with 140 μl of plasma and 560 μl of Buffer AVL, until the tablet was dissolved. Then, lysis was carried out on a Vortex® instrument for 10 minutes under standard conditions with the addition of 560 μl of ethanol. The mixture was added twice to a QIAamp column for binding and centrifuged for 1 minute at 6000×g. Then, for washing with 500 μl of Buffer AW1, centrifugation was carried out for 1 minute at 6000×g, and subsequently with 500 μl of Buffer AW2, centrifugation was carried out for 3 minutes at maximum g-force. After centrifugation for 1 minute at maximum g-force, elution with 80 μl of Buffer TE and centrifugation for 1 minute at 6000×g were carried out.

The subsequent quantitative PCR was carried out with the primers and probes specific for the E. coli phage fr, fr Forward (SEQ ID NO: 7), fr Reverse (SEQ ID NO: 8) and fr Probe (SEQ ID NO: 9), with 12.50 μl of QIAGEN QuantiTect Probe RT-PCR Master Mix 2×, 0.25 μl of QuantiTect RT Mix, 0.10 μl of fr Reverse (100 μM), 0.10 μl of fr Reverse (100 μM), 0.05 μl of fr Probe (100 μM), 10.00 μl of double-distilled water, and 2.00 μl of template aliquots and 2.00 μl of the eluate obtained above. The temperature cycles were 30 minutes at 50° C., 15 minutes at 95° C., and 40×(15 seconds at 95° C., 1 minute at 60° C.). The experiment was carried out 24 times in total.

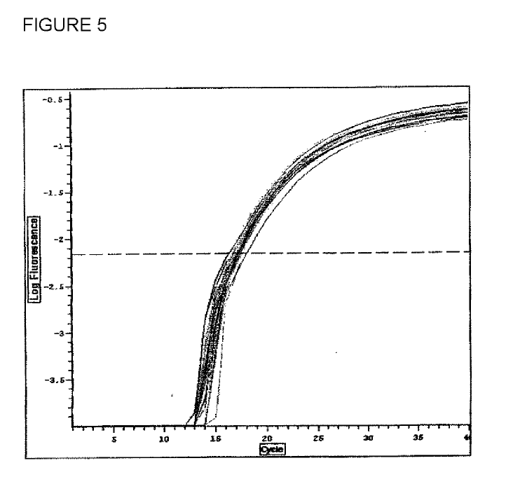

The result is shown in FIG. 5. The figure shows the progression of the fluorescent signal as a function of the number of the PCR cycle. 24 individual samples are shown simultaneously. The mean of the 24 quantitative PCRs is 17.8, and the coefficient of variation (CV) is 2.1%.

The example shows that the specimens according to the invention are outstandingly suitable, in a reproducible manner, for use as an internal standard in methods for RNA preparation and subsequent RNA amplification.

1. A diagnostic reagent in the form of a composition dimensionally stable under standard conditions, comprising at least one bioparticle and at least one pharmaceutical excipient, wherein the bioparticle is selected from the group consisting of bacteria, viruses, fungi, protozoa, bacteriophages, yeasts, spores, parasites, plant cells, animal or human cells, gametes, plasmids, and viroids.

2. An internal standard for methods for nucleic acid preparation and nucleic acid detection comprising a diagnostic reagent as claimed in claim 1.

3. The diagnostic reagent as claimed in claim 1, further comprising at least one buffer substance, detergent, chelating agent, and/or dye.

4. The diagnostic reagent as claimed in claim 1, wherein the pharmaceutical excipient is selected from the group consisting of flow aids, binders, disintegrants, and/or fillers.

5. The diagnostic reagent as claimed in claim 1, additionally comprising at least one of glass particles, zirconium particles, magnetic silica particles, anion exchange resin particles, and/or silicon carbide particles.

6. The diagnostic reagent as claimed in claim 1, additionally comprising one or more enzymes.

7. A method for producing a diagnostic reagent as claimed in claim 1, comprising:
a) mixing said at least one bioparticle and said at least one pharmaceutical excipient at desired amounts
b) portioning on an appropriate carrier material
c) drying.

8. A method for producing a diagnostic reagent as claimed in claim 1, comprising the following steps:
a) mixing elements of said composition at desired amounts, with the exception of the bioparticles
b) portioning on an appropriate carrier material
c) drying
d) applying the bioparticles in the form of a solution onto dried portions
e) drying once more.

9. The method as claimed in claim 7, wherein the drying is carried out each time under standard conditions.

10. A diagnostic reagent produced according to the method of claim 7.

11. An internal standard in nucleic acid preparations with optional subsequent nucleic acid detection comprising a diagnostic reagent as claimed in claim 1.

12. An internal standard as claimed in claim 11 for the quantitative determination of nucleic acids.

13. An internal standard as claimed in claim 11, wherein the nucleic acid preparations are carried out in a microfluidic device.

14. A method for detecting bioparticles, selected from the group consisting of bacteria, viruses, fungi, protozoa, bacteriophages, yeasts, fungi, spores, parasites, plant cells, animal or human cells, gametes, plasmids, and viroids, said method comprising:
withdrawal of sample material
lysis of cells present therein
preparation of released nucleic acids
nucleic acid amplification by PCR
detection of amplified nucleic acids,
wherein a diagnostic reagent as claimed in claim 1 is used as an internal standard in said method.

15. A kit, comprising reagents and vessels required for methods for nucleic acid preparation and/or nucleic acid detection, wherein said kit comprises the diagnostic reagent as claimed in claim 1.

16. The kit as claimed in claim 15, comprising a microfluidic device, reagents required for carrying out nucleic acid preparation and the nucleic acid detection, and the diagnostic reagent as claimed in claim 1.
